(19) United States
(12) Patent Application Publication
(10) Pub. No.: US 2024/0268881 A1
(43) Pub. Date: Aug. 15, 2024

(54) FORCEPS HAVING A SUCTION CUT BLADE

(71) Applicant: GYRUS ACMI, INC.D/B/A OLYMPUS SURGICAL TECHNOLOGIES AMERICA, Westborough, MA (US)

(72) Inventors: Kester Julian Batchelor, Mound, MN (US); Jordan R. Golomb, Maple Grove, MN (US)

(21) Appl. No.: 18/423,796

(22) Filed: Jan. 26, 2024

Related U.S. Application Data
(60) Provisional application No. 63/484,860, filed on Feb. 14, 2023.

(51) Int. Cl. A61B 18/14; A61B 18/00

(52) U.S. Cl. CPC A61B 18/1445; A61B 2018/00184; A61B 2018/00291; A61B 2018/00601; A61B 2018/0063; A61B 2018/1412; A61B 2018/1452

(57) ABSTRACT

A surgical device configured to cut a tissue of a patient can include a first jaw, a second jaw, a cutting blade, and a suction channel. The second jaw can be movably coupled to the first jaw such that the first jaw and the second jaw are moveable between an open position and a closed position. The cutting blade can extend from the second jaw toward the first jaw. The suction channel can be configured to draw the tissue towards the second jaw and to add tension to the tissue and improve one or more cutting capabilities of the surgical device.

### CLAIM OF PRIORITY

This patent application claims the benefit of priority, under 35 U.S.C. Section 119(e), to Kester Batchelor U.S. Patent Application Ser. No. 63/484,860, entitled “FORCEPS HAVING A SUCTION CUT BLADE,” filed on Feb. 14, 2023 (Attorney Docket No. 5409.816PRV), which is hereby incorporated by reference herein in its entirety.

### TECHNICAL FIELD

Examples of the present disclosure generally relate to surgical forceps. More specifically, examples of the present disclosure relate to forceps having a suction cut blade.

### BACKGROUND

Medical devices for diagnosis and treatment, such as forceps, are often used for medical procedures such as laparoscopic and open surgeries. Forceps can be used to manipulate, engage, grasp, or otherwise affect an anatomical feature, such as a vessel or other tissue of a patient during the procedure. Forceps often include an end effector that is manipulatable from a handle of the forceps. For example, jaws located at a distal end of a forceps can be actuated via elements of the handle between open and closed positions to thereby engage the vessel or other tissue. Forceps can include an extendable and retractable blade that can be extended distally between a pair of jaws to lacerate the tissue. Forceps can also include a mechanical blade or electrode blade that can lacerate the tissue. The handle can also be capable of supplying an input energy, such as electromagnetic energy (e.g., radiofrequency energy) or ultrasound, to the end effector for sealing or cutting the vessel or tissue during the procedure.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates a side view of a forceps showing jaws in an open position, in accordance with at least one example of the present disclosure.

FIG. 2 illustrates an isometric view of a portion of a forceps in a closed position, in accordance with at least one example of the present disclosure.

FIG. 3 illustrates an isometric view of a portion of forceps in a partially open position, in accordance with at least one example of the present disclosure.

FIG. 4 illustrates an isometric view of a portion of a forceps in an open position, in accordance with at least one example of the present disclosure.

FIG. 5 illustrates a cross-sectional end view of a portion of a forceps in an open position, taken along line A-A′ in FIG. 2, in accordance with at least one example of the present disclosure.

FIG. 6 illustrates a cross-sectional end view of a portion of a forceps in an open position and surrounding a tissue of a patient, taken along line A-A′ in FIG. 2, in accordance with at least one example of the present disclosure.

FIG. 7 illustrates a cross-sectional end view of a portion of a forceps in a closed position around the tissue of the patient, taken along line A-A′ in FIG. 2, in accordance with at least one example of the present disclosure.

FIG. 8 illustrates a cross-sectional end view of a portion of a forceps in a closed position after sealing the tissue of the patient, taken along line A-A′ in FIG. 2, in accordance with at least one example of the present disclosure.

FIG. 9 illustrates a cross-sectional end view of a portion of a forceps in a closed position after cutting the tissue of the patient, in accordance with at least one example of the present disclosure.

FIG. 10 illustrates a flow diagram of a method for cutting a tissue of a patient, in accordance with at least one example of the present disclosure.

FIG. 11 illustrates a flow diagram of a method for reprocessing a forceps, in accordance with at least one example of the present disclosure.

FIG. 12 is a block diagram illustrating an example of a machine upon which one or more examples may be implemented.

### DETAILED DESCRIPTION

Electrosurgical forceps often include a blade for cutting tissue. In examples, the blade can be a mechanical blade that is positioned within a shaft of the forceps and can be operated form a handle of the forceps to extend between jaws of the forceps to cut tissue therebetween. The mechanical actuation of the blade typically benefits from a stiff shaft because the components therein translate to extend and retract the blade. In some implementations, a forceps can include a flexible shaft or minimization of moving parts, which can be desirable for robot operations. To enable a flexible shaft, the mechanical cutting blade can be replaced with an electrode cutting blade. The inventors of the present disclosure have discovered that tension in the tissue can be improved in forceps, including the electrode-cutting blade, to help cut the tissue with the electrode-cutting blade.

In an example, the forceps can also include sealing plates located on both a first jaw and a second jaw of the forceps. The sealing plates can be used to seal the tissue. The forceps can also include a suction channel within either jaw that can be adjacent to the electrode-cutting blade. The suction channel can be configured to draw the tissue of the patient toward the electrode-cutting blade. The suction channel adjacent to the cutting blade can include a deformable seal that seals the suction channel to prevent suction toward the cutting blade and activates the suction when engaged by the tissue. The electrode-cutting blade can also include a passageway. The passageway can be fluidically connected to the suction channel and the suction channel and the passageway can increase a tension in the tissue throughout the cutting process by drawing the tissue toward the blade.

In an example of the present disclosure, the sealing plates of the forceps can be used to desiccate the tissue and affect the collagen in the tissue to create a seal. The desiccation of the tissue can result in the tissue bulging, and as the tissue bulges, it can deflect the deformable seal to open the suction channel. As such, the suction channel can draw the tissue toward the cutting blade. As the suction channel draws the tissue toward the cutting blade and increases a tension in the tissue at the cutting blade, the electrode cutting blade can be activated to cut the tissue. The passageway in the blade or the suction channel can help continually draw the tissue toward the electrode-cutting blade throughout the cutting process to increase a tension on the tissue and help the electrode-cutting blade cut through the tissue.

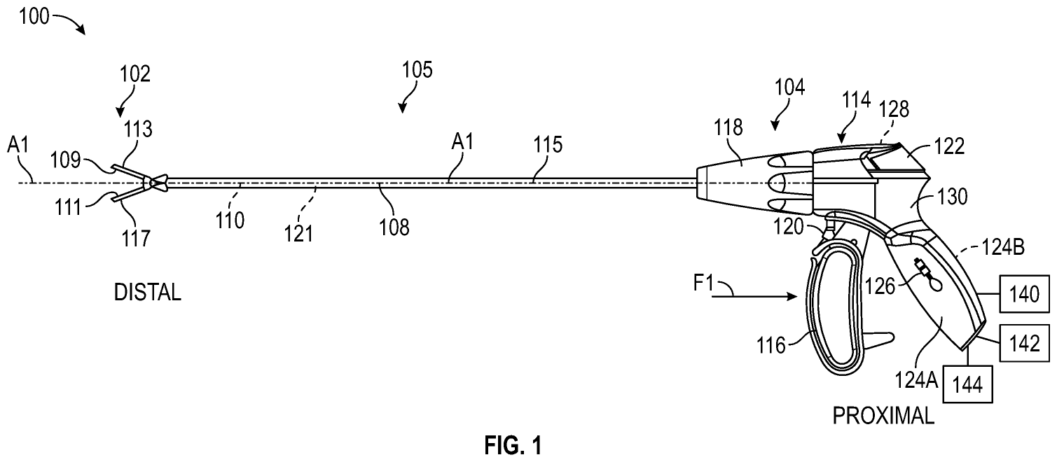

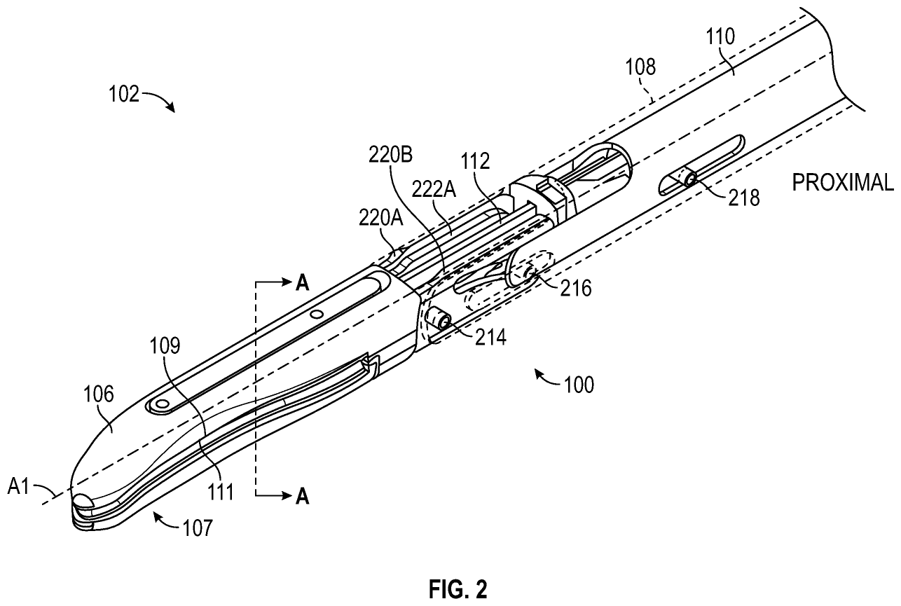

FIG. 1 illustrates a side view of a forceps 100 showing jaws in an open position. Forceps 100 is shown for the purposes of illustration, but features described herein can be used with other forceps, including both handheld and robotic forceps. The illustrative forceps 100 can include an end effector 102, a handpiece 104, and an intermediate portion 105. The end effector 102 can include a first jaw 106, a second jaw 107, an outer shaft 108, a drive shaft 110, and a blade assembly 112 (FIG. 2). The handpiece 104 can include a housing 114, a lever 116, a rotational actuator 118, a trigger 120, an activation button 122, a fixed handle 124A and 124B, and a handle locking mechanism 126. The first jaw 106 can include a first electrode seal plate 109 and the second jaw 107 can include a second electrode seal plate 111. The housing 114 can include a first housing portion 128 and a second housing portion 130. The forceps 100 can also include one or more electrodes (electrodes 115) and a suction channel 121. FIG. 1 also shows orientation indicators Proximal and Distal and a longitudinal axis A1.

Generally, the handpiece 104 can be located at a proximal end of the forceps 100 and the end effector 102 can be located at the distal end of the forceps 100. The intermediate portion 105 can extend between the handpiece 104 and the end effector 102 to operably couple the handpiece 104 to the end effector 102. Various movements of the end effector 102 can be controlled by one or more actuation systems of the handpiece 104. For example, the end effector 102 can be rotated along the longitudinal axis A1 of the forceps 100. Also, the handpiece can operate the first jaw 106, such as by moving the first jaw 106 between open and closed positions. The handpiece 104 can also be used to operate the blade assembly 112 for cutting tissue and can operate the first electrode seal plate 109 for applying electromagnetic energy to tissue. The end effector 102, or a portion of the end effector 102, can be one or more of: opened, closed, rotated, extended, retracted, and electromagnetically energized.

The housing 114 can be a frame that provides structural support between components of the forceps 100. The housing 114 is shown as housing at least a portion of the actuation systems associated with the handpiece 104 for actuating the end effector 102. However, some or all of the actuation components need not be housed within the housing 114. The housing 114 can provide a rigid structure for attachment of components, but the housing 114 does not necessarily house the components completely or can house a portion of one or more of the components.

The electrodes 115 can extend within the intermediate portion 105 between the handpiece 104 and the end effector 102. The electrodes 115 can be used to provide a power source to any of the devices, blades, plates, or the like in the end effector 102. For example, the electrodes 115 can provide a radio frequency energy to the first electrode seal plate 109 (FIG. 2) or the second electrode seal plate 111 (FIG. 2). In another example, the electrodes 115 can provide an electrical current to the first electrode seal plate 109 or the second electrode seal plate 111. Similarly, the electrodes 115 can provide a radiofrequency, electrical, or any other energy source, to the electrode-cutting blade 119 (FIG. 2).

For example, the electrodes 115 can provide energy from a power source 142 (discussed below) to the electrode-cutting blade 119, the first electrode seal plate 109, and the second electrode seal plate 111 to influence how the electrode-cutting blade 119 cuts the tissue and how the first electrode seal plate 109 and the second electrode seal plate 111 seals the tissue. In one example, the energy can be provided so that energy from the electrode-cutting blade 119 travels to the first electrode seal plate 109 or the second electrode seal plate 111 to cut the tissue. The energy can also be provided so that energy from the first electrode seal plate 109 travels to the second electrode seal plate 111 or energy travels from the second electrode seal plate 111 to the first electrode seal plate 109 to seal the tissue.

A proximal portion of the trigger 120 can include or be connected to electronic circuitry within the housing 114 and a distal portion of the trigger 120 can extend outside of the housing 114 adjacent to, and in some examples nested with, the lever 116 in the default or unactuated positions. The activation button 122 can be coupled to the housing 114 and can include or be connected to electronic circuitry within the housing 114. In one example, the trigger 120 and the activation button 122 can be connected to the same electronic circuitry to electronically energize the electrode cutting blade 119 and the electrode sealing plates (109 and 111), respectively. The roles of activation of the trigger 120 and the activation button 122 can be reversed. For example, the trigger 120 can activate the electrode sealing plates and the activation button 122 can activate the electrode cutting blade 119. In another example, a first circuit can communicate with the electrode-cutting blade 119 upon actuation of the trigger 120 and a second circuit can communicate with the sealing plates upon actuation of the activation button 122. Such circuitry can send or transmit electromagnetic energy through forceps 100 to the first jaw 106 or the second jaw 107. In some examples, the electronic circuitry may reside outside the housing 114 but can be operably coupled to the housing 114 and the end effector 102.

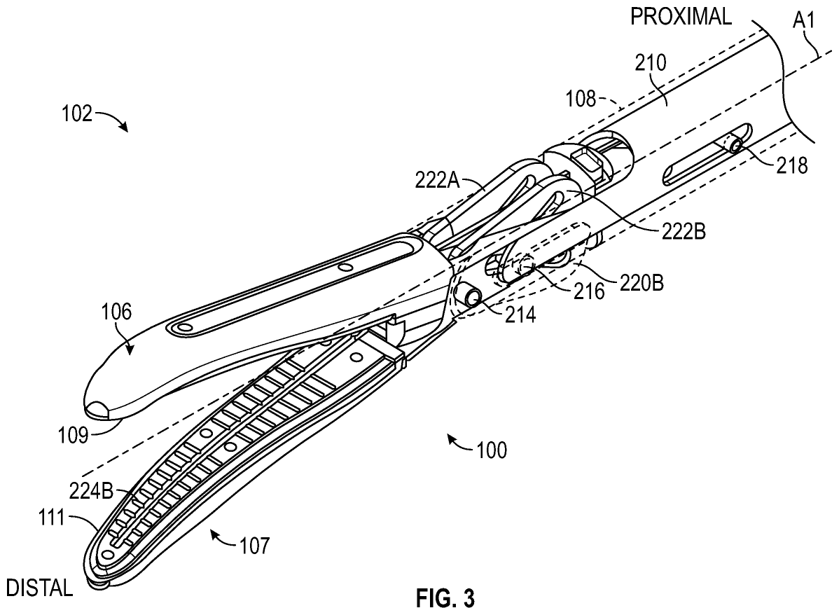

In operation of the forceps 100, a user can displace the lever 116 proximally by applying a Force F1 to the lever 116 to actuate the drive shaft 110 to drive the first jaw 106 from the open position (FIG. 3) to the closed position (FIG. 2), which can allow the user to clamp down on and compress a tissue. The handpiece 104 can also allow a user to rotate the rotational actuator 118 to cause the end effector 102 to rotate, such as by rotating both the drive shaft 110 and the outer shaft 108 together.

In some examples, with the tissue compressed, a user can depress the activation button 122 to cause an electromagnetic energy, or in some examples, ultrasound, to be delivered to the end effector 102, such as to the first electrode seal plate 109, the second electrode seal plate 111, and the tissue. Application of such energy can be used to seal or otherwise affect the tissue being clamped. In some examples, the electromagnetic energy can cause tissue to be coagulated, sealed, ablated, or can cause controlled necrosis. When desired, the trigger 120 can be moved to energize the electrode-cutting blade 119 to cut the tissue within the first jaw 106 and the second jaw 107. Such a process can be repeated, as desired.

The suction channel 121 can fluidically connect the handpiece 104 and the end effector 102. For example, the suction channel 121 can extend in the intermediate portion 105 within the outer shaft 108. The suction channel 121 can be connected to a suction source via the handpiece 104, or the suction channel 121 can include a fitting that extends through the outer shaft 108 to connect to the suction source. The suction channel 121 can be selectively operated such that the suction channel 121 can be on during the entire procedure, or just for a portion of the procedure. For example, the suction channel 121 can be on for the entire procedure, the suction channel can be turned on just prior to, or at the time of, cutting, or the suction channel 121 can be turned on when the tissue deforms the deformable seal after the sealing process. The suction channel 121 can help increase a tension on a tissue during a cutting process of the tissue.

In examples, a suction source 140 can be attached to the forceps 100. For example, the suction source 140 can be attached to handpiece 104 or to any other portion of the forceps 100. The suction source 140 can be fluidically connected to the suction channel 121 to provide suction to the suction channel 121. As will be discussed herein, the suction source 140 can be operated using a controller, manual manipulation, or by manipulation of the deformable seal. For example, a controller can turn on the suction source 140 during the medical procedure just before the cutting will take place. The controller can also automatically turn on the suction source 140. The manual manipulation can be from the operator of the forceps 100 or anyone else in communication with the operator of the forceps 100. For example, the suction source 140 can be manually turned on for the entirety of the procedure, just before cutting, or at any other time during the medical procedure. In another example, the suction source 140 can provide suction force to the suction channel 121 upon deflection or bending of the deformable seal, which will be discussed in more detail herein.

In examples, a power source 142 can be attached to the forceps 100. The power source 142 can provide power to one or more components of the forceps 100, such as to provide energy for sealing, cutting, or any other actions of the forceps 100. For example, the power source 142 can provide electrical, ultrasonic, ultraviolet, radiofrequency, or any other energy source to the components of the forceps 100.

In examples, the forceps 100 can include a controller 144. The controller 144 can include processing circuitry that can control one or more components of the forceps 100. For example, the controller 144 can be used to navigate the forceps 100 within a patient, actuate the forceps 100, control the suction source 140 and the suction channel 121, control the power source 142 and the sealing or cutting of tissue, or any other action, process, or procedure completed by the forceps 100. The controller 144 will be discussed in more detail herein.

In some examples, the forceps 100, or other medical device, may not include all the features described or may include additional features and functions, and the operations may be performed in any order. The handpiece 104 can be used with a variety of other end effectors to perform other methods.

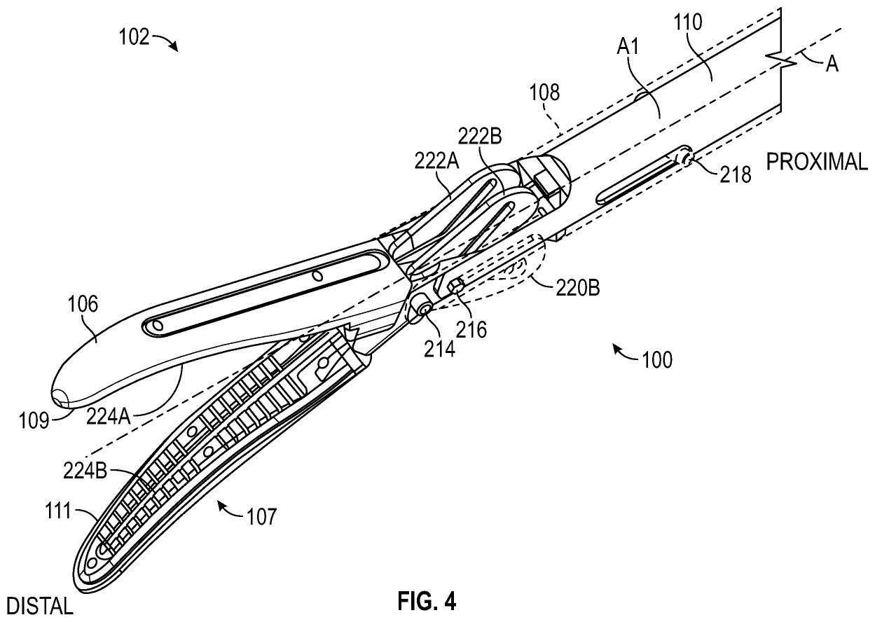

FIG. 2 illustrates an isometric view of a portion of the forceps 100 in a closed position. FIG. 3 illustrates an isometric view of a portion of the forceps 100 in a partially open position. FIG. 4 illustrates an isometric view of a portion of the forceps 100 in an open position. FIGS. 2-4 are discussed below concurrently.

The forceps 100 can include an end effector 102 that can be connected to a handle (such as the handle 104). The end effector 102 can include the first jaw 106 and the second jaw 107, an outer shaft 108, first grip plate 109 and second grip plate 111, an inner shaft 110, a blade assembly 112, a pivot pin 214, a drive pin 216, and a guide pin 218. The first jaw 106 can include flanges 220A and 220B, and the second jaw 107 can include flanges 222A and 222B. The first electrode seal plate 109 can be a grip plate that can include a blade slot 224A and the second electrode seal plate 111 can also be a grip plate that can include a blade slot 224B. FIGS. 2-4 also show orientation indicators Proximal and Distal and a longitudinal axis A1.

Any of the components of the forceps 100 can be comprised of materials such as one or more of metals, plastics, foams, elastomers, ceramics, composites, combinations thereof, or the like. Materials of some components of the forceps 100 are discussed below in further detail.

The first jaw 106 and the second jaw 107 (collectively referred to as jaws 106 and 107) can be rigid or semi-rigid members configured to engage tissue. The jaws 106 and 107 can be coupled to the outer shaft 108, such as pivotably coupled, via the pivot pin 214. The pivot pin 214 can extend through a portion of the jaws 106 and 107 (such as a bore of each of the jaws 106 and 107) such that the pivot pin 214 can be received by outer arms of the outer shaft 108. In other examples, the jaws 106 and 107 can be pivotably coupled to the outer shaft 108. In another example, the outer shaft 108 can include a boss (or bosses) receivable in bores of the outer shaft 108 to pivotably couple the jaws 106 and 107 to the outer shaft 108.

The flanges 220A and 220B (which can be a set of flanges, that is, two flanges) can be rigid or semi-rigid members located at a proximal portion of the first jaw 106. Similarly, the flanges 222A and 222B can be rigid or semi-rigid members located at a proximal portion of the second jaw 107. In some examples, the flanges 220 can be positioned laterally outward of the inner flanges 222. In other examples, the flanges 220 and 222 can be interlaced.

The first electrode seal plate 109 and the second electrode seal plate 111 of the jaws 106 and 107 can each be a rigid or semi-rigid member configured to engage tissue or the opposing jaw to grasp tissue, such as during an electrosurgical procedure. One or more of the first electrode seal plate 109 or the second electrode seal plate 111 can include one or more of serrations, projections, ridges, or the like configured to increase engagement pressure and friction between the first electrode seal plate 109 or the second electrode seal plate 111 and tissue. The flanges 220 of the first jaw 106 can extend proximally away from the first electrode seal plate 109, and in some examples, substantially downward when the first jaw 106 is in the open and partially open positions (as shown in FIGS. 3 and 4, respectively). Similarly, the flanges 222 of the second jaw 107 can extend proximally away from second electrode seal plate 111, and in some examples, substantially upward when the second jaw 107 is in the open and partially open positions (as shown in FIGS. 3 and 4, respectively), such that the jaws 106 and 107 and flanges 220 and 222 can operate to open and close in a scissoring manner.

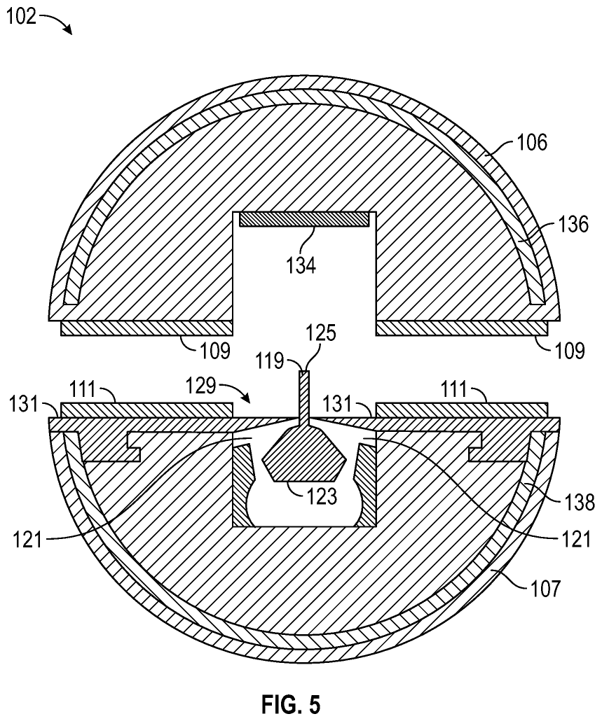

The jaws 106 and 107 can each include an electrode configured to deliver electricity to tissue (optionally through the first electrode seal plate 109 and second electrode seal plate 111), and a frame supporting the electrode. For example, the first jaw 106 can include the first electrode seal plate 109 and the second jaw 107 can include the second electrode seal plate 111. The electrode seal plates 109 and 111 will be discussed in more detail below with reference to FIGS. 5-9.

The blade slots 224A and 224B of the first grip plate first electrode seal plate 109 can each be configured to receive a blade between the jaws 106 and 107, when the jaws are moved out of the open position. In some examples, only one blade slot may be used. In such an example, the opposing blade slot can be configured to receive a compressible member 134 (first shown in FIG. 5).

Each of the inner shaft 110 and the outer shaft 108 can be a rigid or semi-rigid and elongated body having a geometric shape of a cylinder, where the shape of the inner shaft 110 matches the shape of the outer shaft 108. In another example, the outer shaft 108 and the inner shaft 110 can be flexible such that they can more easily be navigated within a patient. In some examples, the inner shaft 110 and the outer shaft 108 can have other shapes such as an oval prism, a rectangular prism, a hexagonal prism, an octagonal prism, or the like. In some examples, the shape of the inner shaft 110 can be different from the shape of the outer shaft 108.

The inner shaft 110 can extend substantially proximally to distally along the axis A1, which can be a longitudinal axis. In some examples, the axis A1 can be a central axis. Similarly, the outer shaft 108 can extend substantially proximally to distally along the axis A1. In some examples, the axis A1 can be a central axis of one or more of the inner shaft 110 and the outer shaft 108. The inner shaft 110 can include an axial bore extending along the axis A1. The outer shaft 108 can also include an axial bore extending along the axis A1. The inner shaft 110 can have an outer dimension (such as an outer diameter) smaller than an inner diameter of the outer shaft 108 such that the inner shaft 110 can be positioned within the outer shaft 108 and such that the inner shaft 110 can be translatable in the outer shaft 108 along the axis A1. The inner shaft 110 can also be referred to as a drive shaft 210, a cam shaft 210, or an inner tube 210. The outer shaft 108 can also be referred to as an outer tube 108.

The guide pin 218, the drive pin 216, and the pivot pin 214 can each be a rigid or semi-rigid pin, such as a cylindrical pin. The guide pin 218, the drive pin 216, and the pivot pin 214 can have other shapes in other examples, such as rectangular, square, oval, or the like. In some examples, the pins can all be of the same size but can be different sizes in other examples. Each pin can have a smooth surface to help reduce surface friction between the pins and components of the forceps 100, such as between the pivot pin 214 and the outer shaft 108 or the drive pin 216 and the flanges 220 and 222. Each of the guide pin 218, the drive pin 216, and the pivot pin 214 can be other components such as one or more projections, bosses, arms, or the like.

In operation, the inner shaft 110 can be translated using an actuator (such as the lever 116 of FIG. 1). The inner shaft 110 can translate with respect to the outer shaft 108 to move the drive pin 216. The drive pin 216 can engage the flanges 220 and 222 to move the flanges 220 and 222 between open and closed positions, which can cause the jaws 106 and 107 to pivot about the pivot pin 214 (such as with respect to the inner shaft 110, the outer shaft 108, or the blade 112) to move the jaws 106 and 107 between open and closed positions.

Features disclosed herein may be used with the illustrative forceps 100 or can be implemented in other forceps. Forceps 100 is provided merely for the purposes of illustration. While aspects, including cutting, suctioning and tissue tensioning, and compressing aspects are described using an example of handheld forceps, aspects described herein may also be incorporated into other handheld forceps and robotic forceps.

FIG. 5 illustrates a cross-sectional end view of a portion of an example end effector 102 that can be provided in forceps 100 shown in an open position. As shown in FIG. 5, the first jaw 106 can include the first electrode seal plate 109, a compressible member 134, and a stiffening member 136. The second jaw 107 can include the second electrode seal plate 111, a blade assembly 112, a suction channel 121, a deformable seal 131, and a stiffening member 138.

The electrode seal plates 109 and 111 can be formed on or can be integral with the grip plates (209A and 209B from FIGS. 2-4). The first electrode seal plate 109 can be on the first face 113 of the first jaw 106. The second electrode seal plate 111 can be on the second face 117 of the second jaw 107. When the first jaw 106 and the second jaw 107 are in the closed position, the first electrode seal plate 109 and the second electrode seal plate 111 can be operable to desiccate the tissue and affect collagen within the tissue to create a seal in the tissue. In some examples, the first electrode seal plate 109 and the second electrode seal plate 111 can be coated with a coating, such as a non-stick coating, hydrophobic coating, nano surface finish, or any other coating that can help prevent the tissue 10 from sticking to the first electrode seal plate 109 and the second electrode seal plate 111, any combination thereof, or the like.

The blade assembly 112 can be configured to cut the tissue of the patient. The blade assembly 112 can include a base 123. In examples, the cutting blade 119 can extend from the base 123 to the distal portion 125. As shown in FIG. 5, the blade assembly 112 can be within the second jaw 107. In another example, the blade assembly 112 can be within the first jaw 106. In examples, the cutting blade 119 can be an electrode-cutting blade 119. As such, the electrode-cutting blade 119 can be in communication with a circuit board within the handpiece 104. In examples, the electrode-cutting blade 119 can include a coating to prevent the tissue 10 from sticking thereto. For example, the electrode-cutting blade 119 can be coated with a non-stick coating, hydrophobic coating, or can have a nanocoating or nano surface finish, or any other coating or treatment that can prevent the tissue 10 from collecting thereon, or the like.

The suction channel 121 can be installed within the second jaw 107 such that the suction channel 121 is adjacent to the blade assembly 112. The suction channel 121 can include an inlet 129 that can be adjacent the base 123 of the blade assembly 112. The suction channel 121 can also include a deformable seal 131 (e.g., resiliently deformable seal). The deformable seal 131 can be configured to seal the inlet 129 of the suction channel 121. The deformable seal 131 can be operable to open the inlet 129 of the suction channel 121 when suction is turned on and can be operable to close the inlet 129 when suction is turned off. Control of the suction can be provided by processing circuitry of the controller 144 (FIG. 1) that is included in or is in communication with the suction source 140 (FIG. 1). In some examples, the vacuum source may provide intermittent or continuous suctioning.

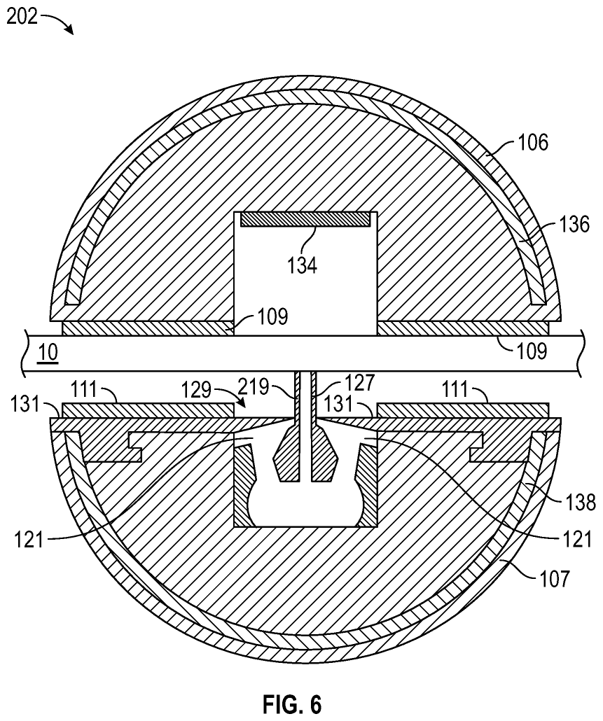

In some examples, the suctioning action can be controlled by the deformable seal 131 being engaged and deflected by the tissue 10 such that the inlet 129 is exposed. When inlet 129 is exposed, the tissue 10 is exposed to the suction and the tissue is caused to be drawn further toward the second jaw 107. Examples of this are further shown and described in the example of FIGS. 6-8. In examples, the suction channel 121 can be connected to a vacuum source (e.g., the suction source 140 from FIG. 1), that can connect to the forceps 100 via the handpiece 104. In another example, the suction channel 121 can be connected to a vacuum source (e.g., the suction source 140 from FIG. 1), that connects the intermediate portion 105 of the forceps 100.

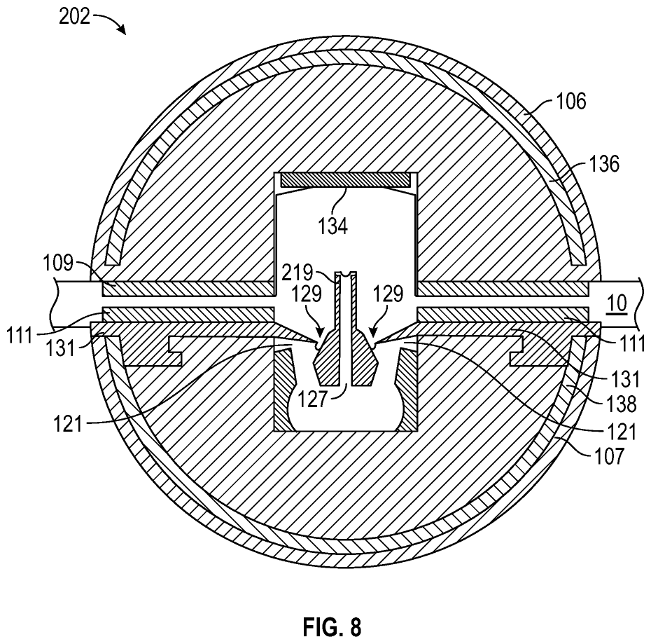

The compressible element 134 can be attached to the first jaw 106 such that the compressible element can be opposite the cutting blade 119 when the first jaw 106 and the second jaw 107 can be in the closed position such that the tissue can be compressed into the compressible element 134 to increase tension on the tissue 10 while cutting with the cutting blade 119. The compressible member 134 will be discussed in more detail below with reference to FIG. 8. In some examples, the compressible member 134 can be omitted.

Each of the first jaw 106 and the second jaw 107 can include the stiffening member 138. The stiffening member 138 can be made from steel, aluminum, titanium, any other rigid material, any combination thereof, or the like. The stiffening member 138 can be configured to provide structure to the first jaw 106 and the second jaw 107. The stiffening member 138 can extend from a proximal portion of the first jaw 106 and the second jaw 107 to a distal portion of the first jaw 106 and the second jaw 107, respectively. The stiffening member 138 can also extend adjacent a periphery of the first jaw 106 and the second jaw 107 to help the first jaw 106 and the second jaw 107 maintain the cross-sectional shape shown in FIG. 5.

FIG. 6 illustrates a cross-sectional end view of a portion of another example end effector 202 of the forceps 100 in an open position and surrounding a tissue of a patient (tissue 10). As shown in FIG. 6, the forceps 100 can be configured to actuate into the open position and surround the tissue 10. In an example, the forceps 100 can navigate into the patient in the closed position, and the operator of the forceps 100 can engage the lever 116 to put the first jaw 106 and the second jaw 107 in the open position once the forceps 100 reaches the tissue 10.

In some examples, and as first shown in FIG. 6, the electrode-cutting blade 219 can also include a passageway 127 extending from the base 123 and through the distal portion 125, the passageway 127 fluidically connected to the suction channel 121 such as to draw the tissue 10 toward the distal portion 125 of the electrode-cutting blade 219. The passageway 127 will be discussed in more detail with reference to FIG. 8 below.

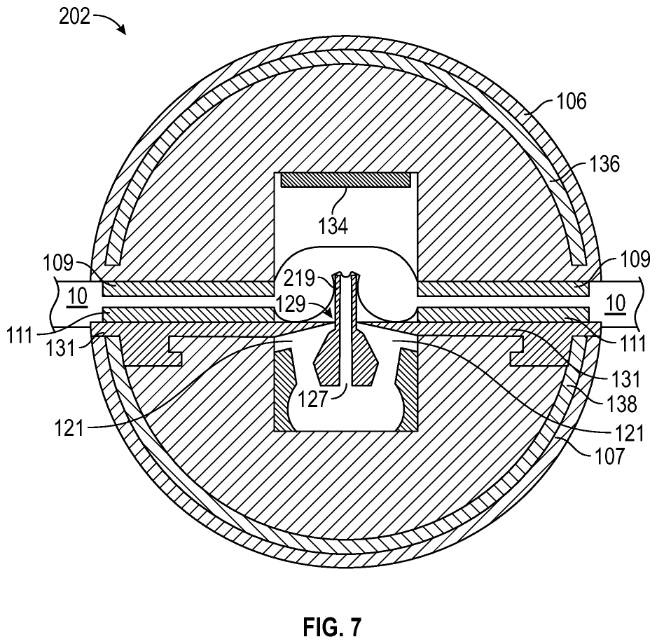

FIG. 7 illustrates a cross-sectional end view of a portion of the end effector 202 in a closed position around the tissue 10. The first jaw 106 and the second jaw 107 can be actuated to move toward the closed position around the tissue 10. As the first jaw 106 and the second jaw 107 move toward the closed position, the forceps 100 can compress the tissue 10. In an example, the first electrode seal plate 109 and the second electrode seal plate 111 can compress the tissue 10 as the first jaw 106 and the second jaw 107 move toward the closed position. As can be seen in FIG. 7, the tissue 10 can bulge between the first jaw 106 and the second jaw 107 as the first jaw 106 and the second jaw 107 move toward the closed position. The tissue 10 can also bulge outside the forceps 100 because of the compressive force applied to the tissue 10 from the first jaw 106 and the second jaw 107.

As the first jaw 106 and the second jaw 107 move toward the closed position, the suction channel 121 in the electrode-cutting blade 219 can draw the tissue 10 toward the second jaw 107. Drawing the tissue 10 toward the second jaw 107 can help hold the tissue 10 between the first jaw 106 and the second jaw 107 as the first jaw 106 and the second jaw 107 moves toward the closed position. In another example, the suction channel 121 can be activated after the first jaw 106 and the second jaw 107 are closed to hold the tissue 10 in the position between the first jaw 106 and the second jaw 107.

In an example, as the first jaw 106 and the second jaw 107 move toward the closed position with the tissue 10 therebetween, the first electrode seal plate 109 and the second electrode seal plate 111 can be activated (via that handpiece 104) to seal the tissue 10. As discussed above, the first electrode seal plate 109 and the second electrode seal plate 111 can desiccate the tissue 10 and affect the collagen in the tissue 10 such as to create a seal in the tissue 10 to prepare the tissue 10 for cutting.

FIG. 8 illustrates a cross-sectional end view of a portion of the end effector 202 in a closed position after sealing the tissue 10. As shown in FIG. 8, as the tissue 10 is sealed, the tissue 10 bulges and expands between the first jaw 106 and the second jaw 107. As the tissue 10 expands between the first jaw 106 and the second jaw 107 the tissue 10 can engage with the deformable seal 131 and the compressible member 134.

The deformable seal 131 can be operable to open the inlet 129 of the suction channel 121 when engaged by the tissue 10 such that the passageway 127 continuously draws the tissue toward the second jaw and the inlet 129 draws the tissue 10 toward the second jaw when the deformable seal 131 can be engaged by the tissue 10 of the patient. Thus, in examples, the suction channel 121 can be on continuously, and only engage with the tissue 10 through the inlet 129 when the deformable seal 131 is actuated to open the inlet 129 of the suction channel 121. The deformable seal 131 can be made from any metallic, polymer, composite, or any combination of flexible or deformable material that can be used as a valve on the inlet 129, or the like.

The compressible member 134, if provided, can be configured to compress to increase a pressure on the tissue 10. The compressible member 134 can be attached to the first jaw 106 such that the compressible member 134 is opposite of the electrode-cutting blade 219. The compressible member 134 can compress and the compressive force can help direct the tissue 10 toward the second jaw 107. The compressible member 134 can be configured such that the compressible member 134 can be compressed 25 to 300 times before the compressible member 134 needs to be replaced. The compressible member 134 can be made of resilient material such that the compressible member 134 can be replaced after 50 or more activations, or even 250 or more compression cycles.

In examples, the compressible member 134, or if not present, a surface of the first jaw 106, can compress and the compressive force can direct the tissue 10 toward the deformable seal 131 to open and activate the suction channel 121 at the inlet 129 and thereby further draw the tissue 10 toward the second jaw 107. The compressible member 134 can also compress to apply a compressive force on the tissue 10 to direct the tissue 10 toward the electrode-cutting blade 119. The compressible member 134 can be made from any polymers, such as a polymer that includes silicon, a dielectric ceramic, a composite, or any combination of flexible or deformable material that can be compressed to exert an elastic force on the tissue 10, or the like. Therefore, the compressible member 134 can be made of a material that includes electrical insulation to insulate the first electrode seal plate 109 from the second electrode seal plate 111. However, in some examples the compressible member can be conductive, such as to conduct electrical energy to an adjacent electrode.

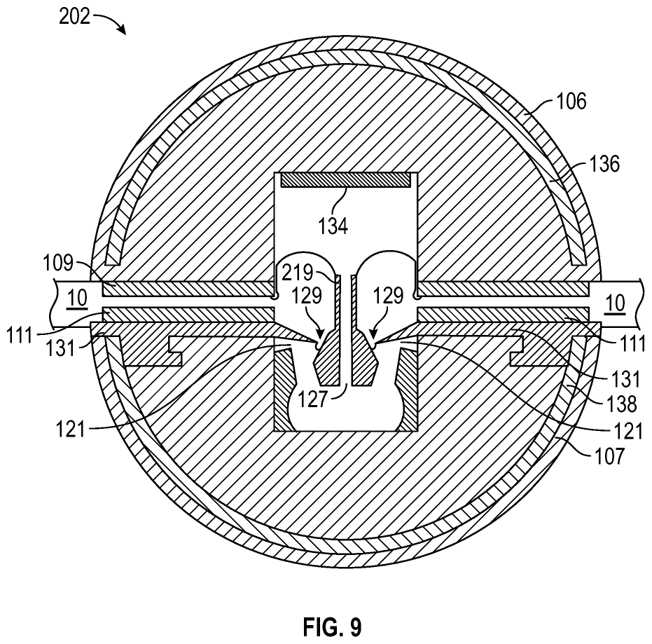

FIG. 9 illustrates a cross-sectional end view of a portion of the end effector 202 in a closed position after cutting the tissue 10. As can be seen in FIG. 9, the tissue 10 is cut, and the end effector 202 can hold onto the tissue at the point of sealing (e.g., first electrode seal plate 109 and second electrode seal plate 111) until the suction is removed from the suction channel 121 and the passageway 127 respectively. As discussed herein, the suction power of the suction channel 121 and the passageway 127 can be controlled by the deformable seal 131, or a control or other mechanism connected to the forceps 100.

The electrodes 115 (FIG. 1) can be used to provide a power source to any of the devices, blades, plates, or the like in the end effector 102. For example, the electrodes 115 can provide a radio frequency energy to the first electrode seal plate 109 (FIG. 2) or the second electrode seal plate 111 (FIG. 2). In another example, the electrodes 115 can provide an electrical current to the first electrode seal plate 109 or the second electrode seal plate 111. Similarly, the electrodes 115 can provide a radiofrequency, electrical, or any other energy source, to the electrode-cutting blade 119 (FIG. 2).

For example, the electrodes 115 can provide energy from a power source 142 (discussed below) to the electrode-cutting blade 119, the first electrode seal plate 109, and the second electrode seal plate 111 to influence how the electrode-cutting blade 119 cuts the tissue and how the first electrode seal plate 109 and the second electrode seal plate 111 seals the tissue. In one example, the energy can be provided so that energy from the electrode-cutting blade 119 travels to the first electrode seal plate 109 or the second electrode seal plate 111 to cut the tissue. The energy can also be provided so that energy from the first electrode seal plate 109 travels to the second electrode seal plate 111 or energy travels from the second electrode seal plate 111 to the first electrode seal plate 109 to seal the tissue.

For example, the forceps 100 can be a bimodal electronic cutting system where the electrode 115 can provide power to the electrode-cutting blade 119 such that the electrode-cutting blade 119 can be a primary source of power. The power within the electrode-cutting blade 119 can then travel to, and in examples can arc from the electrode-cutting blade 119 to, either the first electrode seal plate 109, the second electrode seal plate 111, or both the first electrode seal plate 109 and the second electrode seal plate 111. As the power travels from the electrode-cutting blade 119 to the first electrode seal plate 109 or the second electrode seal plate 111, the tissue 10 can be cut or sealed.

As shown in FIG. 9, the electrode-cutting blade 119 can be a blade that extends from a base and having a passageway extending therethrough. In another example, the electrode-cutting blade 119 can include a folded metal foil that can include perforations formed in a distal portion. The metal foil can be titanium, aluminum, stainless steel, any combination or alloy thereof, or any other material that is electrically conductive and can be used to cut the tissue 10. The perforations in the distal portion can be fluidically connected to the passageway 127 to draw the tissue 10 toward the electrode-cutting blade 119 throughout the cutting process of the tissue.

As shown in FIG. 9, the suction channel 121 can extend adjacent to the electrode-cutting blade 119 and include the deformable seal 131 to control the inlet 129. In another example, the suction channel 121 can fluidically connect to a proximal portion of the electrode-cutting blade 119 and the areas adjacent to the electrode-cutting blade 119 can be fluidically isolated from the suction channel 121 to prevent fluidic connection to the suction channel 121. Here, a distal portion of electrode-cutting blade 119 can provide the only fluidic connection to the suction channel 121 via the passageway 127 to draw the tissue 10 toward the second jaw 107 and provide tension in the tissue 10 throughout the cutting process.

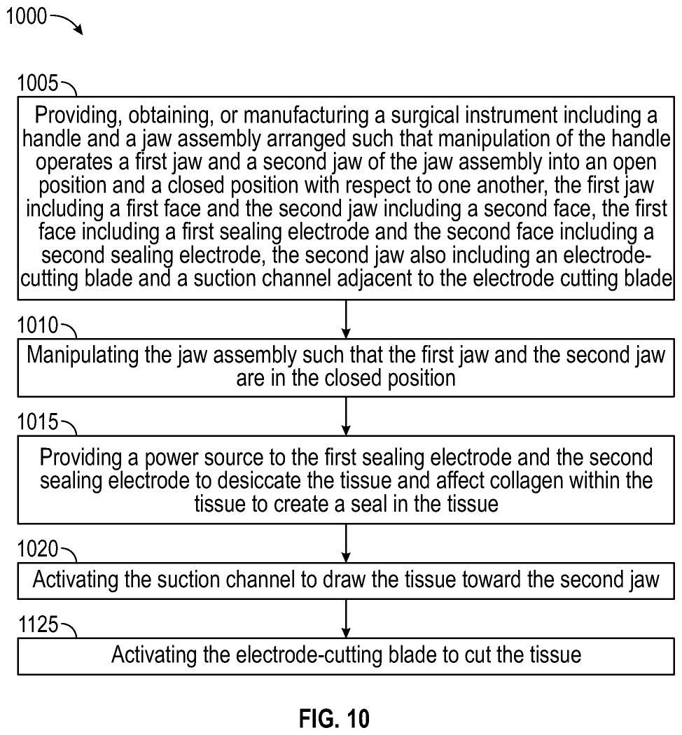

FIG. 10 illustrates a schematic view of a method 1000, in accordance with at least one example of this disclosure. The method 1000 can be a method of sealing and cutting tissue of a patient. More specific examples of the method 1000 are discussed below. The steps or operations of the method 1000 are illustrated in a particular order for convenience and clarity; many of the discussed operations can be performed in a different sequence or in parallel without materially impacting other operations. However, in some examples, the steps of the method are performed in the order shown. The method 1000 as discussed includes operations performed by multiple different actors, devices, or systems. It is understood that subsets of the operations discussed in the method 1000 can be attributable to a single actor, device, or system and could be considered a separate standalone process or method. The method 1000 can be performed with example forceps described herein, however, the method 1000 can also be performed with other forceps, and the forceps described herein can be used with different methods.

In an example, at procedure 1005, the method 1000 can include providing, obtaining, or manufacturing a surgical instrument including a handle and a jaw assembly arranged such that manipulation of the handle operates a first jaw and a second jaw of the jaw assembly into an open position and a closed position with respect to one another. The first jaw can include a first face and the second jaw can include a second face. The first face can include a first sealing electrode and the second face can include a second sealing electrode. The second jaw can also include an electrode-cutting blade and a suction channel adjacent to the electrode cutting blade.

In an example, at procedure 1010, the method 100 can include manipulating the jaw assembly such that the first jaw and the second jaw can be in the closed position. As discussed above, the first jaw and the second jaw can be closed around the tissue such as to compress the tissue and get the first face closer to the second face. The proximity of the first face and the second face permit the electrical transfer between the first sealing electrode and the second sealing electrode while sealing the tissue of the patient.

At procedure 1015, the method 1000 can include providing a power source to the first sealing electrode and the second sealing electrode to desiccate the tissue and affect collagen within the tissue to create a seal in the tissue. As discussed above, the desiccation of the tissue can cause the tissue to bulge. As the tissue bulges, it can engage with a deformable seal on an inlet of the suction channel to connect the inlet and the suction source.

At procedure 1020, the method 1000 can include activating the suction channel to draw the tissue toward the second jaw. For example, the suction channel can be turned on to provide a suction at the inlet of the suction channel adjacent the cutting blade. The suction can help draw the tissue of the patient into the cutting blade to increase a tension in the tissue and help cut the tissue of the patient. In an example, the cutting blade can also include a passageway that can help to draw the tissue toward the cutting blade while the cutting blade is cutting the tissue.

At procedure 1025, the method 1000 can include activating the electrode-cutting blade to cut the tissue. Here, the electrode-cutting blade can be activated to cut the tissue. In the examples discussed here, the cutting blade is an electrode cutting blade. In another example, the suction channel can be used to support the cutting of an optical cutting source, an ultrasonic cutting source, a resistive cutting source, a laser cutting source or a mechanical cutting blade. For example, the suction channel can be adjacent the cutting source, e.g., the laser or the mechanical blade assembly. As such, any of the features of the discussed example device can be used for any of the various devices used to cut tissue of a patient. The discussed components can also be used on other medical devices, such as for example, a medical stapler, which can have a suction channel adjacent to the stapler to draw the tissue toward the staple. In the examples presented here, the device includes a jaw that can pivot to open and close. In examples, the devices discussed herein can be used in a medical device that remains open. For example, a cutting device that does not include jaws and has a mechanical blade that extends and retracts can include a suction channel adjacent to the blade in the extended location such as to draw the tissue of the patient toward the cutting mechanism of the device.

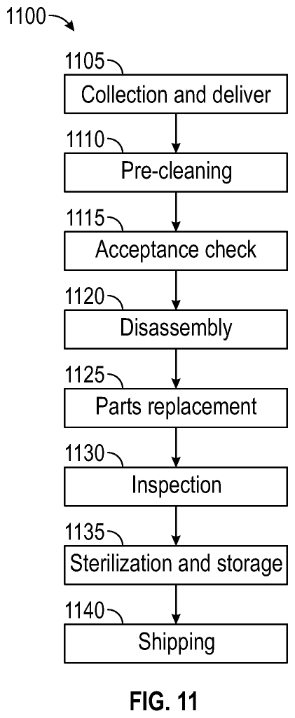

Next, the reprocessing method for the above-described forceps 100 will be described. FIG. 11 is a flowchart indicating a reprocessing method 1100 for the forceps 100. The forceps 100 described above can be disposed of after one use, or can be repeatedly used a plurality of times. In the case of a configuration that is repeatedly used a plurality of times, for example, reprocessing method shown in FIG. 11 can be used. An operator who remanufactures collects the used forceps 100 after it has been used for treatment and transports it to a factory or the like (Step 1105). At this time, the used forceps 100 can be transported in a dedicated container to prevent contamination from the forceps 100.

Then, the operator can clean and sterilize the collected and transported used forceps 100 (Step 1110). Specifically, in cleaning the forceps 100, deposits adhering to the end effector 102, first jaw 106, second jaw 107, or any other part of the forceps 100, can be removed by using a brush or the like. After that, to remove pathogenic microorganisms and the like derived from blood, body fluid, etc., the components of the forceps 100 can be used with any cleaning solution of isopropanol-containing cleaning agent, proteolytic enzyme detergent, and alcohol. And the components can be cleaned. The cleaning liquid is not limited to the cleaning liquid described above, and other cleaning liquids may be used. Further, in the sterilization of the forceps 100, to sterilize the pathogenic microorganisms and the like adhering to the end effector 102, first jaw 106, second jaw 107, or any other part of the forceps 100, any of high-pressure steam sterilization, ethylene oxide gas sterilization, gamma ray sterilization, hydrogen peroxide, and hydrogen peroxide low temperature sterilization can be used. The end effector 102 is made of the first jaw 106 and the second jaw 107, which are connected by the pivot pin 214. Therefore, the end effector 102, first jaw 106, second jaw 107 can be easy to clean.

And the operator performs an acceptance check of the used forceps 100 (Step 1115). In detail, the operator checks whether the used forceps 100 has significant defects or the used forceps 100 exceed a maximum number of reprocessing.

Next, in some examples, the operator can disassemble the used forceps 100 (Step 1115). For example, the operator can remove the pivot pin 214 to detach the flanges 220 and the flanges 222 to disassemble the end effector 102. In some examples, the electrodes 115 can also be disconnected from the first electrode seal plate 109, second electrode seal plate 111, and the electrode-cutting blade 119.

After the step 1115, some parts can be replaced (Step 1120). For example, the compressible member 134 or the deformable seal 131 can be replaced if the compressible member 134, such as if the compressible member 134 or deformable seal 131 is consumed by treatment, or as a standard step. In addition, a cleaning or sterilizing step can be performed while the compressible member 134 or the deformable seal 131 are removed, before being replaced to allow cleaning or sterilizing of hard to reach surfaces. In some examples, removal can be performed before step 1110. After completion of step 1115, the result is a distinct end effector or forceps from the original forceps 100.

After step 1120, if the pivot pin 214 was removed, or the end effector otherwise detached from the forceps, the operator reassembles the end effector to the forceps 100 (e.g., handpiece), or assembles the end effector to a new forceps (Step 1125) either way, the result is creating a distinct forceps. For example, the new forceps 100 can be assembled such that the end effector 102 is formed by putting the pivot pin 214 between the flanges 220 and the flanges 222.

In some examples, Step 1125 can include adding an identifier to indicate the device has been modified from its original condition, such as adding a label or other marking to designate the device as reprocessed, refurbished or remanufactured.

After step 1125, the operator inspects and tests the newly formed forceps, now distinct from forceps 100 (step 1130). Specifically, the operator who remanufactures verifies that the newly formed forceps 100 has the same effectiveness and safety as the original product by various functional tests.

After Step 1130, the operator sequentially performs a sterilization and storage (Step 1135), and shipping (Step 1140) of the new forceps 100. In the Step 1140, in step 1145, a sterilization treatment using a sterilizing gas such as ethylene oxide gas or propylene oxide gas is applied to the new forceps 100 and the device can be stored in a storage container until use.

Steps 1105 to 1140 described above can be executed to achieve reprocessing of the forceps 100.

The devices disclosed herein can be designed to be disposed of after a single use, or they can be designed to be used multiple times. In either case, however, the device can be reconditioned for reuse after at least one use. Reconditioning can include a combination of the steps of disassembly of the device, followed by cleaning or replacement of particular pieces, and subsequent reassembly. In particular, the device can be disassembled, and any number of particular pieces or parts of the device can be selectively replaced or removed in any combination. For example, the electrodes can be disconnected from the first electrode seal plate 109, the second electrode seal plate 111, and the electrode-cutting blade 119 such that the end effector 102 can be removed from the forceps 100 and the first jaw 106 can be separated from the second jaw 107.

Upon cleaning or replacement of particular parts, the device can be reassembled for subsequent use either at a reconditioning facility, or by a surgical team immediately prior to a surgical procedure. Those of ordinary skill in the art will appreciate that the reconditioning of a device can utilize a variety of different techniques for disassembly, cleaning/replacement, and reassembly. Use of such techniques, and the resulting reconditioned device, are all within the scope of the present application.

Preferably, the invention described herein will be processed before surgery. First a new or used instrument is obtained and, if necessary, cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or higher energy electrons. The radiation kills bacteria on the instrument and in the container. The sterilized instrument can then be stored in the sterile container. The sealed container keeps the instrument sterile until it is opened in the medical facility. The device may also be sterilized using any other technique known in the art, including but limited to beta or gamma radiation, ethylene oxide, or steam.

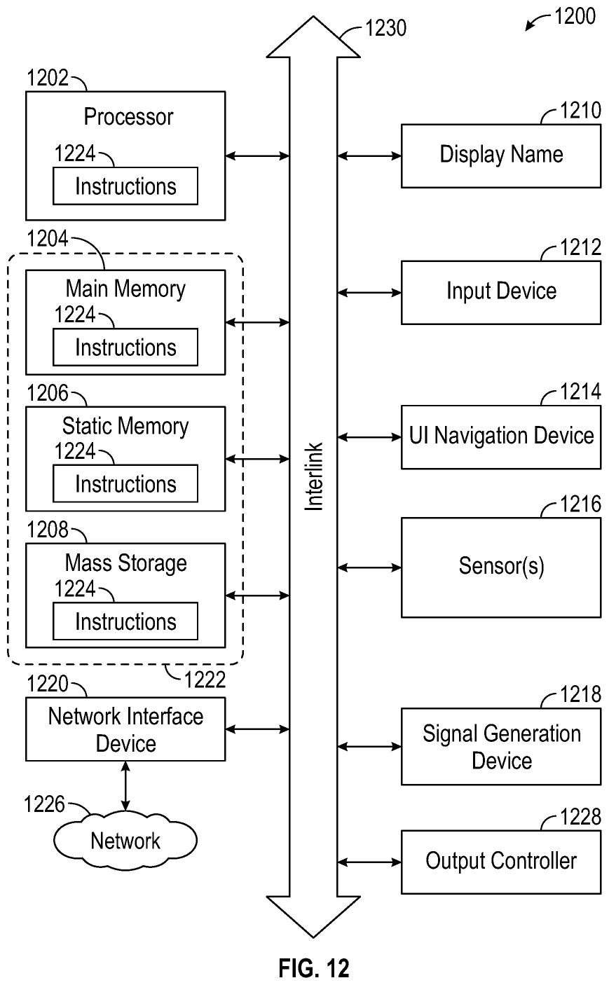

FIG. 12 illustrates a block diagram of an example machine Error! Reference source not found.00 upon which any one or more of the techniques (e.g., methodologies) discussed herein may perform. Examples, as described herein, may include, or may operate by, logic or a number of components, or mechanisms in the machine Error! Reference source not found.00. Circuitry (e.g., processing circuitry) is a collection of circuits implemented in tangible entities of the machine Error! Reference source not found.00 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine Error! Reference source not found.00 follow.

In alternative embodiments, the machine Error! Reference source not found.00 may operate as a standalone device or may be connected (e.g., networked) to other machines. In a networked deployment, the machine Error! Reference source not found.00 may operate in the capacity of a server machine, a client machine, or both in server-client network environments. In an example, the machine Error! Reference source not found.00 may act as a peer machine in peer-to-peer (P2P) (or other distributed) network environment. The machine Error! Reference source not found.00 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine (e.g., computer system) Error! Reference source not found.00 may include a hardware processor Error! Reference source not found.02 (e.g., a central processing unit (CPU), a graphics processing unit (GPU), a hardware processor core, or any combination thereof), a main memory Error! Reference source not found.04, a static memory (e.g., memory or storage for firmware, microcode, a basic-input-output (BIOS), unified extensible firmware interface (UEFI), etc.) Error! Reference source not found.06, and mass storage Error! Reference source not found.08 (e.g., hard drives, tape drives, flash storage, or other block devices) some or all of which may communicate with each other via an interlink (e.g., bus) Error! Reference source not found.30. The machine Error! Reference source not found.00 may further include a display unit Error! Reference source not found. 10, an alphanumeric input device Error! Reference source not found. 12 (e.g., a keyboard), and a user interface (UI) navigation device Error! Reference source not found. 14 (e.g., a mouse). In an example, the display unit Error! Reference source not found. 10, input device Error! Reference source not found. 12 and UI navigation device Error! Reference source not found. 14 may be a touch screen display. The machine Error! Reference source not found.00 may additionally include a storage device (e.g., drive unit) Error! Reference source not found.08, a signal generation device Error! Reference source not found. 18 (e.g., a speaker), a network interface device Error! Reference source not found.20, and one or more sensors Error! Reference source not found. 16, such as a global positioning system (GPS) sensor, compass, accelerometer, or other sensor. The machine Error! Reference source not found.00 may include an output controller Error! Reference source not found.28, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor Error! Reference source not found.02, the main memory Error! Reference source not found.04, the static memory Error! Reference source not found.06, or the mass storage Error! Reference source not found.08 may be, or include, a machine readable medium Error! Reference source not found.22 on which is stored one or more sets of data structures or instructions Error! Reference source not found.24 (e.g., software) embodying or utilized by any one or more of the techniques or functions described herein. The instructions Error! Reference source not found.24 may also reside, completely or at least partially, within any of registers of the processor Error! Reference source not found.02, the main memory Error! Reference source not found.04, the static memory Error! Reference source not found.06, or the mass storage Error! Reference source not found.08 during execution thereof by the machine Error! Reference source not found.00. In an example, one or any combination of the hardware processor Error! Reference source not found.02, the main memory Error! Reference source not found.04, the static memory Error! Reference source not found.06, or the mass storage Error! Reference source not found.08 may constitute the machine readable media Error! Reference source not found.22. While the machine readable medium Error! Reference source not found.22 is illustrated as a single medium, the term “machine readable medium” may include a single medium or multiple media (e.g., a centralized or distributed database, and/or associated caches and servers) configured to store the one or more instructions Error! Reference source not found.24.

The term “machine readable medium” may include any medium that is capable of storing, encoding, or carrying instructions for execution by the machine Error! Reference source not found.00 and that cause the machine Error! Reference source not found.00 to perform any one or more of the techniques of the present disclosure, or that is capable of storing, encoding or carrying data structures used by or associated with such instructions. Non-limiting machine readable medium examples may include solid-state memories, optical media, magnetic media, and signals (e.g., radio frequency signals, other photon based signals, sound signals, etc.). In an example, a non-transitory machine readable medium comprises a machine readable medium with a plurality of particles having invariant (e.g., rest) mass, and thus are compositions of matter. Accordingly, non-transitory machine-readable media are machine readable media that do not include transitory propagating signals. Specific examples of non-transitory machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

In an example, information stored or otherwise provided on the machine readable medium Error! Reference source not found.22 may be representative of the instructions Error! Reference source not found.24, such as instructions Error! Reference source not found.24 themselves or a format from which the instructions Error! Reference source not found.24 may be derived. This format from which the instructions Error! Reference source not found.24 may be derived may include source code, encoded instructions (e.g., in compressed or encrypted form), packaged instructions (e.g., split into multiple packages), or the like. The information representative of the instructions Error! Reference source not found.24 in the machine readable medium Error! Reference source not found.22 may be processed by processing circuitry into the instructions to implement any of the operations discussed herein. For example, deriving the instructions Error! Reference source not found.24 from the information (e.g., processing by the processing circuitry) may include: compiling (e.g., from source code, object code, etc.), interpreting, loading, organizing (e.g., dynamically or statically linking), encoding, decoding, encrypting, unencrypting, packaging, unpackaging, or otherwise manipulating the information into the instructions Error! Reference source not found.24.

In an example, the derivation of the instructions Error! Reference source not found.24 may include assembly, compilation, or interpretation of the information (e.g., by the processing circuitry) to create the instructions Error! Reference source not found.24 from some intermediate or preprocessed format provided by the machine readable medium Error! Reference source not found.22. The information, when provided in multiple parts, may be combined, unpacked, and modified to create the instructions Error! Reference source not found.24. For example, the information may be in multiple compressed source code packages (or object code, or binary executable code, etc.) on one or several remote servers. The source code packages may be encrypted when in transit over a network and decrypted, uncompressed, assembled (e.g., linked) if necessary, and compiled or interpreted (e.g., into a library, stand-alone executable etc.) at a local machine, and executed by the local machine.

The instructions Error! Reference source not found.24 may be further transmitted or received over a communications network Error! Reference source not found.26 using a transmission medium via the network interface device Error! Reference source not found.20 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), LoRa/LoRaWAN, or satellite communication networks, mobile telephone networks (e.g., cellular networks such as those complying with 3G, 4G LTE/LTE-A, or 5G standards), Plain Old Telephone (POTS) networks, and wireless data networks (e.g., Institute of Electrical and Electronics Engineers (IEEE) 802.11 family of standards known as Wi-Fi®, IEEE 802.15.4 family of standards, peer-to-peer (P2P) networks, among others. In an example, the network interface device Error! Reference source not found.20 may include one or more physical jacks (e.g., Ethernet, coaxial, or phone jacks) or one or more antennas to connect to the communications network Error! Reference source not found.26. In an example, the network interface device Error! Reference source not found.20 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine Error! Reference source not found.00, and includes digital or analog communications signals or other intangible medium to facilitate communication of such software. A transmission medium is a machine readable medium.

The following, non-limiting examples, detail certain aspects of the present subject matter to solve the challenges and provide the benefits discussed herein, among others.

Example 1 is a surgical device for cutting a tissue of a patient, the surgical device comprising: a first jaw having a first face; a second jaw movably coupled to the first jaw such that the first jaw and the second jaw are moveable between an open position and a closed position, the second jaw having a second face that is substantially adjacent to the first face when the first jaw and the second jaw are in the closed position; a cutting blade extending from the second jaw toward the first jaw; and a suction channel within the second jaw, the suction channel configured to draw the tissue towards the second jaw to add tension to the tissue and improve one or more cutting capabilities of the surgical device.

In Example 2, the subject matter of Example 1 includes, wherein the cutting blade includes an electrode-cutting blade.

In Example 3, the subject matter of Example 2 includes, wherein the electrode-cutting blade comprises: a base connected to the second jaw; a distal portion, the electrode-cutting blade extending from the base to the distal portion; and a passageway extending from the base and through the distal portion, the passageway fluidically connected to the suction channel such as to draw the tissue toward the distal portion of the electrode-cutting blade.

In Example 4, the subject matter of Example 3 includes, a first electrode seal plate on the first face; and a second electrode seal plate on the second face; wherein on condition that the first jaw and second jaw are in the closed position, the first electrode seal plate and the second electrode seal plate are operable to desiccate the tissue and affect collagen within the tissue to create a seal in the tissue.

In Example 5, the subject matter of Example 4 includes, wherein the suction channel comprises: an inlet adjacent to and around the electrode-cutting blade; and a deformable seal configured to seal the inlet of the suction channel, the deformable seal operable to open the inlet of the suction channel when engaged by the tissue such that the passageway continuously draws the tissue toward the second jaw and the inlet draws the tissue toward the second jaw when the deformable seal is engaged by the tissue of the patient.

In Example 6, the subject matter of Examples 2-5 includes, wherein the electrode-cutting blade uses radio frequency to cut the tissue.

In Example 7, the subject matter of Examples 1-6 includes, a first electrode seal plate on the first face; and a second electrode seal plate on the second face; wherein on condition that the first jaw and second jaw are in the closed position, the first electrode seal plate and the second electrode seal plate are operable to desiccate the tissue and affect collagen within the tissue to create a seal in the tissue.

In Example 8, the subject matter of Example 7 includes, wherein the suction channel comprises: an inlet adjacent to and around the cutting blade; and a deformable seal configured to seal the inlet of the suction channel, the deformable seal operable to open the inlet of the suction channel when engaged by the tissue such that the inlet of the suction channel draws the tissue toward the second jaw when the tissue engages with the deformable seal.

In Example 9, the subject matter of Examples 1-8 includes, a compressible element attached to the first jaw such that the compressible element is opposite the cutting blade when the first jaw and the second jaw are in the closed position such that the tissue can be compressed into the compressible element to increase tension on the tissue while cutting with the cutting blade.

Example 10 is a surgical device configured to cut a tissue of a patient, the surgical device comprising: a first jaw; a second jaw movably coupled to the first jaw such that the first jaw and the second jaw are moveable between an open position and a closed position; a cutting blade extending from the second jaw toward the first jaw; and a suction channel within the second jaw, the suction channel configured to draw the tissue towards the second jaw to add tension to the tissue and improve one or more cutting capabilities of the surgical device.

In Example 11, the subject matter of Example 10 includes, wherein the cutting blade includes an electrode-cutting blade.

In Example 12, the subject matter of Example 11 includes, wherein the electrode-cutting blade comprises: a base connected to the second jaw; a distal portion, the electrode-cutting blade extending from the base to the distal portion; and a passageway extending from the base and through the distal portion, the passageway fluidically connected to the suction channel such as to draw the tissue toward the distal portion of the electrode-cutting blade.

In Example 13, the subject matter of Example 12 includes, wherein the first jaw comprises a first face and the second jaw comprises a second face, and wherein the second face is substantially adjacent to the first face when the first jaw and the second jaw are in a closed position.

In Example 14, the subject matter of Example 13 includes, a first electrode seal plate on the first face; and a second electrode seal plate on the second face; wherein on condition that the first jaw and second jaw are in the closed position, the first electrode seal plate and the second electrode seal plate are operable to desiccate the tissue and affect collagen within the tissue to create a seal in the tissue.

In Example 15, the subject matter of Example 14 includes, wherein the suction channel comprises: an inlet adjacent to and around the electrode-cutting blade; and a deformable seal configured to seal the inlet of the suction channel, the deformable seal operable to open the inlet of the suction channel when engaged by the tissue such that the passageway continuously draws the tissue toward the second jaw and the inlet draws the tissue toward the second jaw when the tissue of the patient engages the deformable seal.

In Example 16, the subject matter of Examples 10-15 includes, wherein the first jaw comprises a first face and the second jaw comprises a second face, and wherein the second face is substantially adjacent to the first face when the first jaw and the second jaw are in the closed position.

In Example 17, the subject matter of Example 16 includes, a first electrode seal plate on the first face; and a second electrode seal plate on the second face; wherein on condition that the first jaw and second jaw are in the closed position, the first electrode seal plate and the second electrode seal plate are operable to desiccate the tissue and affect collagen within the tissue to create a seal in the tissue.

In Example 18, the subject matter of Example 17 includes, wherein the suction channel comprises: an inlet adjacent to and around the cutting blade; and a deformable seal configured to seal the inlet of the suction channel, the deformable seal operable to open the inlet of the suction channel when engaged by the tissue such that the inlet of the suction channel draws the tissue toward the second jaw when the tissue engages with the deformable seal.

Example 19 is a method for cutting a tissue of a patient, the method comprising: providing, obtaining, or manufacturing a surgical instrument including a handle and a jaw assembly arranged such that manipulation of the handle operates a first jaw and a second jaw of the jaw assembly into an open position and a closed position with respect to one another, the first jaw including a first face and the second jaw including a second face, the first face including a first sealing electrode and the second face including a second sealing electrode, the second jaw also including an electrode-cutting blade and a suction channel adjacent to the electrode cutting blade; manipulating the jaw assembly such that the first jaw and the second jaw are in the closed position; providing a power source to the first sealing electrode and the second sealing electrode to desiccate the tissue and affect collagen within the tissue to create a seal in the tissue; activating the suction channel to draw the tissue toward the second jaw; and activating the electrode-cutting blade to cut the tissue.

In Example 20, the subject matter of Example 19 includes, wherein activating the suction channel to draw the tissue toward the second jaw, comprises: turning on the suction channel; and engaging, with the tissue, a deformable seal installed adjacent to the suction channel, the deformable seal operable to activate and deactivate the suction channel from drawing the tissue toward the second jaw.

Example 21 is an apparatus comprising means to implement of any of Examples 1-20.

Example 22 is a system to implement of any of Examples 1-20.

Example 23 is a method to implement of any of Examples 1-20.

Example 24 is a method of reprocessing the device of any of Examples 1-20, the method can include obtaining the device of any of claims 1-20; sterilizing the device, and storing the device in a sterile container to prepare the device for shipping.

The above-detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

1. A surgical device for cutting a tissue of a patient, the surgical device comprising:
a first jaw having a first face;
a second jaw movably coupled to the first jaw such that the first jaw and the second jaw are moveable between an open position and a closed position, the second jaw having a second face that is substantially adjacent to the first face when the first jaw and the second jaw are in the closed position;
a cutting blade extending from the second jaw toward the first jaw; and
a suction channel within the second jaw, the suction channel configured to draw the tissue towards the second jaw to add tension to the tissue and improve one or more cutting capabilities of the surgical device.

2. The surgical device of claim 1, wherein the cutting blade includes an electrode-cutting blade.

3. The surgical device of claim 2, wherein the electrode-cutting blade comprises:
a base connected to the second jaw;
a distal portion, the electrode-cutting blade extending from the base to the distal portion; and
a passageway extending from the base and through the distal portion, the passageway fluidically connected to the suction channel such as to draw the tissue toward the distal portion of the electrode-cutting blade.

4. The surgical device of claim 3, comprising:
a first electrode seal plate on the first face; and
a second electrode seal plate on the second face;
wherein on condition that the first jaw and second jaw are in the closed position, the first electrode seal plate and the second electrode seal plate are operable to desiccate the tissue and affect collagen within the tissue to create a seal in the tissue.

5. The surgical device of claim 4, wherein the suction channel comprises:
an inlet adjacent to and around the electrode-cutting blade; and
a deformable seal configured to seal the inlet of the suction channel, the deformable seal operable to open the inlet of the suction channel when engaged by the tissue such that the passageway continuously draws the tissue toward the second jaw and the inlet draws the tissue toward the second jaw when the deformable seal is engaged by the tissue of the patient.

6. The surgical device of claim 2, wherein the electrode-cutting blade uses radio frequency to cut the tissue.

7. The surgical device of claim 1, comprising:
a first electrode seal plate on the first face; and
a second electrode seal plate on the second face;
wherein on condition that the first jaw and second jaw are in the closed position, the first electrode seal plate and the second electrode seal plate are operable to desiccate the tissue and affect collagen within the tissue to create a seal in the tissue.

8. The surgical device of claim 7, wherein the suction channel comprises:
an inlet adjacent to and around the cutting blade; and
a deformable seal configured to seal the inlet of the suction channel, the deformable seal operable to open the inlet of the suction channel when engaged by the tissue such that the inlet of the suction channel draws the tissue toward the second jaw when the tissue engages with the deformable seal.

9. The surgical device of claim 1, comprising:
a compressible element attached to the first jaw such that the compressible element is opposite the cutting blade when the first jaw and the second jaw are in the closed position such that the tissue can be compressed into the compressible element to increase tension on the tissue while cutting with the cutting blade.

10. A surgical device configured to cut a tissue of a patient, the surgical device comprising:
a first jaw;
a second jaw movably coupled to the first jaw such that the first jaw and the second jaw are moveable between an open position and a closed position;
a cutting blade extending from the second jaw toward the first jaw; and
a suction channel within the second jaw, the suction channel configured to draw the tissue towards the second jaw to add tension to the tissue and improve one or more cutting capabilities of the surgical device.

11. The surgical device of claim 10, wherein the cutting blade includes an electrode-cutting blade.

12. The surgical device of claim 11, wherein the electrode-cutting blade comprises:
a base connected to the second jaw;
a distal portion, the electrode-cutting blade extending from the base to the distal portion; and
a passageway extending from the base and through the distal portion, the passageway fluidically connected to the suction channel such as to draw the tissue toward the distal portion of the electrode-cutting blade.

13. The surgical device of claim 12, wherein the first jaw comprises a first face and the second jaw comprises a second face, and wherein the second face is substantially adjacent to the first face when the first jaw and the second jaw are in a closed position.

14. The surgical device of claim 13, comprising:
a first electrode seal plate on the first face; and
a second electrode seal plate on the second face;
wherein on condition that the first jaw and second jaw are in the closed position, the first electrode seal plate and the second electrode seal plate are operable to desiccate the tissue and affect collagen within the tissue to create a seal in the tissue.

15. The surgical device of claim 14, wherein the suction channel comprises:
an inlet adjacent to and around the electrode-cutting blade; and
a deformable seal configured to seal the inlet of the suction channel, the deformable seal operable to open the inlet of the suction channel when engaged by the tissue such that the passageway continuously draws the tissue toward the second jaw and the inlet draws the tissue toward the second jaw when the tissue of the patient engages the deformable seal.

16. The surgical device of claim 10, wherein the first jaw comprises a first face and the second jaw comprises a second face, and wherein the second face is substantially adjacent to the first face when the first jaw and the second jaw are in the closed position.

17. The surgical device of claim 16, comprising:
a first electrode seal plate on the first face; and
a second electrode seal plate on the second face;
wherein on condition that the first jaw and second jaw are in the closed position, the first electrode seal plate and the second electrode seal plate are operable to desiccate the tissue and affect collagen within the tissue to create a seal in the tissue.

18. The surgical device of claim 17, wherein the suction channel comprises:
an inlet adjacent to and around the cutting blade; and
a deformable seal configured to seal the inlet of the suction channel, the deformable seal operable to open the inlet of the suction channel when engaged by the tissue such that the inlet of the suction channel draws the tissue toward the second jaw when the tissue engages with the deformable seal.

19. A method for cutting a tissue of a patient, the method comprising:
providing, obtaining, or manufacturing a surgical instrument including a handle and a jaw assembly arranged such that manipulation of the handle operates a first jaw and a second jaw of the jaw assembly into an open position and a closed position with respect to one another, the first jaw including a first face and the second jaw including a second face, the first face including a first sealing electrode and the second face including a second sealing electrode, the second jaw also including an electrode-cutting blade and a suction channel adjacent to the electrode-cutting blade;
manipulating the jaw assembly such that the first jaw and the second jaw are in the closed position;
providing a power source to the first sealing electrode and the second sealing electrode to desiccate the tissue and affect collagen within the tissue to create a seal in the tissue;
activating the suction channel to draw the tissue toward the second jaw; and
activating the electrode-cutting blade to cut the tissue.

20. The method of claim 19, wherein activating the suction channel to draw the tissue toward the second jaw, comprises:
turning on the suction channel; and
engaging, with the tissue, a deformable seal installed adjacent to the suction channel, the deformable seal operable to activate and deactivate the suction channel from drawing the tissue toward the second jaw.
